SEQUENCE LISTING (18 sequences)

SEQ ID NO: 1 - protein, 140 aa, human
MPSAFSVSSFPVSIPAVLTQTDWTEPWLMGLATFHALCVLLTCLSSRSYRLQIGHFLCLVILVYCAEYINEAAAMNWRLFSKYQYFDSRGMFISIVFSAPLLVNAMIIVVMWVWKTLNVMTDLKNAQERRKEKKRRRKED

SEQ ID NO: 2 - protein, 122 aa, mouse
METDWTEPWLLGLLAFHLLCLLLTCFSSQRYKLQIGHFLCLVVLVYSAEYINEVAAVNWRLFSKYQYFDSRGMFISLVFSAPLLFNAMLIVIMWVRKTLTVMTDLKTLQEERKERRRRRKEE

SEQ ID NO: 3 - protein, 140 aa, rat
MASPYSVRVFPVSIPAVIMETDWTEPWLLGLLAFHLLCLLLTCFSAQRYKLQIGHFLCLVVLVYCAEYINEVAAMNWRLFAKYQYFDSRGMFISLVFSAPLLFNAMVIVIMWVRKTLTVMSDLKNLQERRKERKRRRKEE

SEQ ID NO: 4 - protein, 411 aa, dog
MCEWCCWQRPYAYLLSARDDFRCEGYGGPTKLRAPSLCITAPVEAACARVADPLSSSNGRLPARHRERSPRNFKLTGDVSVQRGLEHRKTGGKLPSQEARVPACLLLHHRGREGAADTLPQDHEWAVFCEWRLQERLARARARTTGASVTVPIPAILGVHRPVTQTHRVSPSGLAVAQRPPRSQELCGLQHPARERSGGDALHERVVPRPGACCHLLPPGETATGPVHTGSPTPVRLGTRAGQESGPGPNTRDTSHLRGAPFRNPTCRDAVPPPWGPLLRRVLPRLRLGPRPQTDWTEPWLLGLAVFHVLCLLLTCLSSQRYKLQVGHFLCLVILVYCAEYINEIAAMNWRLFSKYQYFDSRGMFISIVFSAPLLLNAMIIVILWVRKTLNVMTDLKTLQEKRRERKRKEE

SEQ ID NO: 5 - protein, 159 aa, chicken
MVSLAIWASSREASAVRMRSVAVAMEQPLHGPPGLSTILARTDWAEPWLLGLAGFHVLCFLLTCFSFQHYRVQIGHFLCMVCLVYCAEYINELAAMNWRLFSKYQYFDSRGMFISLVFSAPLLVNTIIIVVNWVYRTLNVMTELKTLQQRIKAEKDKKK

SEQ ID NO: 6 - DNA, 23 bp, artificial
  synthetic primer
caccatgccgtccgccttctctg

SEQ ID NO: 7 - DNA, 25 bp, artificial
  synthetic primer
aaagtcttctttccttctccttttc

SEQ ID NO: 8 - DNA, 19 bp, artificial
  synthetic primer
atgccgtccgccttctctg

SEQ ID NO: 9 - DNA, 22 bp, artificial
  synthetic primer
gtcttctttccttctccttttc

SEQ ID NO: 10 - DNA, 19 bp, artificial
  synthetic primer
tcatgtttgagaccttcaa

SEQ ID NO: 11 - DNA, 19 bp, artificial
  synthetic primer
gtctttgcggatgtccacg

SEQ ID NO: 12 - DNA, 25 bp, artificial
  synthetic primer
tcatcttagtctactgtgctgaata

SEQ ID NO: 13 - DNA, 25 bp, artificial
  synthetic primer
tgctcacgcagacggactggactga

SEQ ID NO: 14 - protein, 14 aa, artificial
  synthetic peptide derived from TMEM18 sequence
DLKNAQERRKEKKR

SEQ ID NO: 15 - DNA, 31 bp, artificial
  synthetic primer
cagcgcaaaaaacgccgccagcgccgctaga

SEQ ID NO: 16 - DNA, 31 bp, artificial
  synthetic primer
ctagcggcgctggcggcgttttttgcgctga

SEQ ID NO: 17 - DNA, 20 bp, artificial
  synthetic primer
tcagtcttctttccttctcc

SEQ ID NO: 18 - DNA, 20 bp, artificial
  synthetic primer
aagaatgcacaagagagaag

(19) United States
(12) Patent Application Publication
(10) Pub. No.: US 2010/0111913 A1
(43) Pub. Date: May 6, 2010

(54) METHOD OF ENHANCING MIGRATION OF NEURAL PRECURSOR CELLS

(75) Inventors: Shu Wang, Singapore (SG); Jaana Jurvansuu, Singapore (SG)

Correspondence Address: KLARQUIST SPARKMAN, LLP, 121 SW SALMON STREET, SUITE 1600, PORTLAND, OR, 97204, US

(73) Assignee: Agency for Science, Technology and Research

(21) Appl. No.: 12/532,825

(22) PCT Filed: Mar. 20, 2008
(86) PCT No.: PCT/SG2008/000089

§ 371 Date: Jan. 19, 2010

Related U.S. Application Data
(60) Provisional application No. 60/907,187, filed on Mar. 23, 2007.

(51) Int. Cl. A61K 45/00; C12N 5/071; C12N 5/079; A61P 25/00; A61P 25/28

(52) U.S. Cl. 424/93.21; 435/325; 424/93.7

(57) ABSTRACT

There is provided methods relating to enhancing migration of a neural precursor cell by augmenting levels of TMEM18 protein in the neural precursor cell. Such methods may be used in the treatment of disorders, including treatment of a glioma or a neurodegenerative disorder.

### CROSS-REFERENCE TO RELATED APPLICATION

This application claims benefit of, and priority from, U. S. provisional patent application No. 60/907,187, filed on Mar. 23, 2007, the contents of which are fully incorporated herein by reference.

### FIELD OF THE INVENTION

The present invention relates generally to methods of delivering neural progenitor cells to sites within neural tissue.

### BACKGROUND OF THE INVENTION

Gliomas are the most common type of intracranial tumours, and have a tendency to invade rapidly in the brain. Gliomas originate from glial cells, predominantly from astrocytes, and are graded from I to IV with increasing level of malignancy. Grade IV gliomas, also named glioblastoma multiforme (GBM), comprise nearly half of gliomas and are the most frequent primary brain tumours in adults. GBM is currently almost incurable. Even with surgery, radiotherapy and chemotherapy, patients with GBM usually die within about a year, with only a few patients surviving longer than 3 years. Thus, glioma treatments and therapies have become a major focus for cancer therapy.

Gliomas are among the most lethal of all cancers and do not respond well to current therapies. The high lethality of gliomas can be attributed to the ability of glioma cells to infiltrate surrounding healthy brain tissue. Glioma cells exploit misregulation of growth factors, proteases, and extracellular matrix and cell surface proteins to gain their devastating invasion capacity (reviewed in Tysnes et al., 2001; Mueller et al., 2003).

The failure of current glioma therapies is mainly due to this ability of the tumour cells to extensively invade the surrounding healthy brain tissue, hence escaping localised treatments. As localised treatments are thus inefficient and comprehensive treatments are too damaging to the delicate brain, a preferred solution is to find a treatment that can specifically locate the tumour cells.

### SUMMARY OF THE INVENTION

In one aspect, there is provided a method of enhancing migration of a neural precursor cell toward a cell that secretes a neural precursor cell chemoattractant factor, the method comprising augmenting levels of TMEM18 in the neural precursor cell.

In another aspect, there is provided a neural precursor cell having augmented levels of TMEM18.

In another aspect, there is provided a pharmaceutical composition comprising a neural precursor cell having augmented levels of TMEM18.

In another aspect, there is provided use of neural precursor cell having augmented levels of TMEM18 for treating a neural disorder, including use of neural precursor cell having augmented levels of TMEM18 in the preparation of a medicament for treating a neural disorder.

In various embodiments of the different aspect of the present invention, the TMEM18 may comprise an amino acid sequence as set forth in any one of SEQ ID NO.: 1 to SEQ ID NO.: 5.

The neural precursor cell may be derived from an embryonic stem cell or from neural tissue. The neural precursor cell may be modified to include a marker molecule or a therapeutic agent. The neural precursor cell may comprise an expression vector encoding TMEM18, and may comprise an expression vector encoding a therapeutic protein or peptide. The neural precursor cell may accordingly be used for treating a neural disorder.

The cell that secretes a neural precursor chemoattractant factor may be a glioma cell or a cell at a site of neural tissue degeneration.

The neural precursor chemoattractant factor may comprise Stromal cell Derived Factor, Monocyte Chemoattractant Protein, Cytokine Stem Cell Factor, Epidermal Growth Factor, Vascular Endothelial Growth Factor, Stem Cell Factor or Fibroblast Growth Factor.

Augmenting levels of TMEM18 may comprise increasing expression levels of native TMEM18 in the neural precursor cell or expressing recombinant TMEM18 from an expression vector in the neural precursor cell.

The presently described methods may further comprise delivering the neural precursor cell to a cell population containing the cell that secretes the neural precursor chemoattractant factor. The cell population may be in vitro or in vivo. Delivering may include surgical implantation or injection.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention:

FIG. 1: Identifying TMEM18 as a gene regulating the migration of neural precursor cells towards glioma cells. (A) Flowchart of cDNA library screen to identify proteins able to promote neural precursor cell migration towards glioma cells. Human neural precursor cell line, NT2, was infected with retrovirus carrying a cDNA library. The infected cells were plated on transwell migration insert on top of glioma cell culture. The neural precursor cells able to migrate through the insert's pores to the opposite site of the insert's membrane were collected and passed two more times through the same migration assay. The virus-imported cDNAs in migrated cells were cloned by PCR and sequenced. (B) TMpred-program predicts TMEM18 to have four membrane spanning alpha-helixes (Hofman and Stoffel, 1993). Schematic interpretation of the TMEM18 protein structure is overlaid with TMpred-graph to illustrate the results from the program. (C) Sequence alignment of TMEM18 proteins from human [SEQ ID NO. 1], mouse [SEQ ID NO. 2], rat [SEQ ID NO. 3], dog [SEQ ID NO. 4], and chicken [SEQ ID NO. 5]. An asterix (*) indicates perfect amino acid conservation and a colon (:) one amino acid difference in the sequences. Note that only a C-terminal portion of [SEQ ID NO.: 4] is shown for the dog sequence. Bold underlined sequence represents possible nuclear localization signal peptides predicted by PredictNLS-program (Cokol et al. 2000).

FIG. 2: Stable neural precursor cell lines overexpressing TMEM18. Lentivirus imported TMEM18 overexpression was examined by regular RT-PCR (A, D) and by quantitative real-time RT-PCR (B, E) in NT2 cells and C17.2 cells. Results are shown for non-infected, parental cells (ctrl), empty virus infected control cells (vector control), and for two populations (TMEM18 A and B) for each type of the two neural precursor cells. For real-time RT-PCR analysis, expression of TMEM18 mRNA was normalised using β-actin mRNA expression. (C) Immunostaining to show overexpression of TMEM18 in NT2 cells.

FIG. 3: TMEM18 overexpression increases the migration activity of neural precursor cells. (A, B) The results of scratch assays performed to assess the effects of TMEM18 on nonspecific cell movement. NT2 (A) and C17.2 (B) cells were plated at same densities and a layer of cells was removed by scraping with a pipet tip. Recovery of the empty area was examined 24 hours post-scraping. (C, D) Transwell migration assays to assess the effects of TMEM18 on glioma-specific migrations of neural precursor cells. (C) NT2 cell migrations towards U87 and H4 glioma cell lines and NIH3T3 and 293T non-glioma cell lines. (D) C17.2 cell migrations towards U87 and C6 glioma cell lines. Results are presented as fold differences compared with cells transduced with control viruses. Statistical comparison is calculated between cells overexpressing TMEM18 and vector controls using students T-test. One asterisk (*), two asterisks (**) and three asterisks (***) denote p-values less than 0.10, 0.05 and 0.01, respectively.

FIG. 4: TMEM18 overexpression increases the migration activity of C17.2 neural precursor cells towards C6 glioma cells in the rat brain. Green fluorescent dye-labeled TMEM18 over-expressing C17.2 cells and red fluorescent dye-labeled vector control C17.2 cells were mixed and injected into the left side the rat brain contralateral to a C6 glioma inoculation site (right). Two weeks later, the brains were sectioned for cell migration examination under a florescence microscope. The squares on the right side in A and B indicate the front of cell migration towards the tumour, which were shown in a high magnification in C and D. E is the merged picture of C and D. Dots with yellow color present green and red fluoresce dye-labeled cells moving together. Note many of green fluoresce dye-labeled TMEM18 over-expressing C17.2 cells (arrows) migrating alone.

FIG. 5: Knockdown of endogenous TMEM18 expression inhibits cell migration. (A, B) Short interfering RNAs against TMEM18 transcripts reduced the expression of endogenous TMEM18 in NT2 and C17.2 cells, as quantified by real-time PCR. Two different sequences of short interfering RNAs against TMEM18 were tested in NT2 cells and yielded four different knock-down levels of the TMEM18 mRNA. siRNA against luciferase was used as a control. (C, D) Decrease in TMEM18 expression levels reduced the migration activity of neural precursor cells towards both DMEM and U87MG.

FIG. 6: TMEM18 protein shows localization pattern covering nuclear and cytoplasmic structures. (A) The specificity of produced antibody against TMEM18 peptide was studied by immunofluorescence experiment using pre-immunization serum (Pre-serum), and TMEM18 antibody serum in overexpressing cell lines, and non-infected and empty virus-infected control NT2 cells. TMEM18 is shown with rhodamine and DNA with DAPI. (B) For more accurate TMEM18 protein localization in cells, NT2 cells were stained against TMEM18 (1), DNA (2), and alpha-tubulin (4). Overlay between TMEM18 and DNA (3) reveals that TMEM18 antibody recognises nuclear membrane (a ring around the nucleus), whereas overlay between TMEM18 and alpha-tubulin (5) shows that TMEM18 is located also outside nucleus, into cytoplasm. A superimposition of all TMEM18, DNA, and alpha-tubulin is shown in (6). (C) GFP fusion protein with N-terminus of TMEM18 localises to nucleus. U87 cells were transfected with plasmid vector expressing GFP, HIV TAT linked GFP, or TMEM18 N-terminus linked GFP. Light microscope pictures on left side and fluorescence microscope pictures on right of the same cells.

### DETAILED DESCRIPTION

Neural stem and precursor cells are able to migrate to the sites of brain tumours, making them attractive tools for glioma therapy.

In efforts to improve the glioma tracing ability of neural precursor cells, the present inventors performed a cDNA library expression screen to identify candidate genes that once over-expressed would increase the tropism of neural precursor cells for gliomas. A functionally unannotated gene encoding Transmembrane Protein 18 (TMEM18) was identified as one such gene. The inventors then discovered that over-expression of TMEM18 in neural precursor cells increased the ability of these cells to migrate towards glioma cells, both in vitro and in vivo. The present invention exploits such a discovery to provide methods of enhancing the migration of neural precursor cells.

TMEM18 is a small protein of 140 amino acids in humans, and is predicted to be a transmembrane protein with a nuclear localisation signal. Overexpression of TMEM18 provides neural precursor cells with an enhanced migration capacity toward glioma cells or cells that secrete a neural precursor cell chemoattractant factor.

TMEM18 was first identified in a screen to discover new expressed full length open reading frames (Hofmann and Stoffel, 1993). The inventors are aware of no other publications on this protein since. Now, the inventors have identified TMEM18 as an important factor for neural precursor cell motility. Functional inactivation of the endogenous TMEM18 gene in neural precursor cells results in loss of migration activity of these cells. Thus, it appears that expression of TMEM18 is required for the mobility of neural precursor cells and overexpression of this gene can be favourably used to enhance the tropism of these cells for gliomas. Particularly, TMEM18 acts as a specific enhancer for glioma-directed migration.

The functional importance of TMEM18 is highlighted in the strong amino acid conservation throughout mammals and even reaching to lower forms of multicellular eukaryotes. Furthermore, according to the profile of TMEM18 expressed sequence tags, the protein is transcribed in most of the adult human tissues and in embryonic developmental state (as found in a search of NCBI's EST expression profile viewer).

As stated above, neural precursor cells have an intrinsic tropism for sites of brain injuries including gliomas. As shown first by Benedetti et al. and Aboody et al., engrafted primary and immortalised neuronal precursor cells can be used in gene therapy of gliomas in animal models (Benedetti et al., 2000; Aboody et al, 2000). These engrafted neural precursor cells have been shown to spread through the existing migratory pathways in healthy brain as well as non-typical routes when gliomas are present (Aboody et al, 2000; Flax et al., 1998). Besides primary and immortalised neural stem cells, embryonic stem cell-derived neural progenitor cells seem to have the same aptitude for glioma cell tracking (Arnhold et al., 2003). Moreover, neural stem cells are able to locate not only gliomas but also tumours of non-neuronal origin, which suggests that this tracking ability may be dependent on a general factor secreted at tumour sites (Brown et al., 2004; Allport et al., 2004).

The cues that guide neural precursor cells to gliomas have been studied. Obvious candidate signals to attract neural precursor cells to the sites of brain injuries and tumours are factors released in inflammation. The immunoreactive cell type found in the brain, migroglia, has been shown to release factors which mobilise neural precursor cells (Aarum et al., 2003). Cytokines are small proteins released in inflammation, which are expressed by all cell types in brain including microglia. Cytokines have been shown to provide cues for neural stem cell migration in brain development (reviewed in Tran and Miller, 2003). Stromal cell Derived Factor 1 (SDF-1) chemokine has been shown to attract neural stem cells and when SDF-1's receptor CX Chemokine Receptor 4 was blocked it hindered the neural stem cell migration to the site of injury (Allport et al., 2004; Ehtesham et al., 2004; Imitola et al., 2004). Also, chemokine Monocyte Chemoattractant Protein-1 (MCP-1), the expression of which can be induced by Tumour Necrosis Factor-alpha, can also activate migration of neural stem cells (Widera et al., 2004). Cytokine Stem Cell Factor, which is expressed by glioma cell lines and overexpressed in neurons at the sites of brain injury, is also a contributing attractant for neural stem cells (Erlandsson et al., 2004; Sun et al., 2004; Serfozo et al., 2006). Similarly as for the glioma cell migration, growth factors promote also neural precursor cell migration, as it has been shown that Epidermal Growth Factor Receptor and Vascular Endothelial Growth Factor mediated signaling can increase neural stem cell migration (Boockvar et al., 2003; Schmidt et al., 2005). Glioma invasion depends largely on the cells' ability to modify the extracellular matrix and interestingly it has been shown that the glioma cell line secreted extracellular matrix is able to promote neural stem cell motility as well (Ziu et al., 2006).

Thus, there is provided a method of enhancing the migration of a neural precursor cell. The method involves augmenting the levels of the TMEM18 protein in a neural precursor cell. The neural precursor cell may then be delivered into a region where there is a cell population that may include a cell that secretes a neural precursor cell chemoattractant factor; including a glioma cell.

As used herein, the term “neural precursor” cell is used interchangeably with the terms “neural progenitor” cell and “neural stem” cell and refers to a cell that is not fully differentiated to a particular neural cell type, but which may have the potential to become a neural cell type. Thus, a neural precursor cell as used herein refers to a cell that is undifferentiated or partially undifferentiated, and which can divide and proliferate to produce undifferentiated or partially undifferentiated cells or which can differentiate to produce one or two differentiated or specialized neural cells. Neural precursor cell includes an undifferentiated stem cell, including an embryonic-derived stem cell, that can divide to produce two undifferentiated cells or one undifferentiated cell and one differentiated cell or a partially differentiated precursor cell that can divide to produce two partially differentiated cells or two differentiated cells. A neural precursor cell may be multipotent, which means that the cell is capable of self-renewal and of trans-differentiation into several types of neural cells upon differentiation.

The precursor cell can be obtained from a variety of sources including, but not limited to, neural tissue, neuronal tissue, as well as embryonic cells including embryonic stem cells. The precursor cell can be derived from any animal, including a mammal, and particularly from a rodent or a human. The precursor cells used in the methods of the invention, when used to treat a subject, may be subject derived (autologous) or from a donor of the same species (allogeneic) or of a different species (xenogeneic). If the precursor cell is xenogeneic, preferably the cell is a nude cell, which is a cell that has been modified to not express, or to have reduced or minimal expression of, surface antigens that would induce an immune response in the subject being treated.

The term “cell” as used herein refers to and includes a single cell, a plurality of cells or a population of cells where context permits, unless otherwise specified. The cell may be an in vitro cell including a cell explanted from a subject or it may be an in vivo cell. Similarly, reference to “cells” also includes reference to a single cell where context permits, unless otherwise specified.

Augmentation of TMEM18 levels in a neural precursor cell enhances the ability of the neural precursor cell to migrate toward a glioma cell. The glioma cell may be any glioma cell, including a grade I-IV glioma, including a glioblastoma such as a glioblastoma multiforme.

As discussed above, cell motility relies in part on the cell moving along a chemoattractant gradient. Thus, without being limited to any particular theory, TMEM18 appears to contribute to the ability of a neural precursor cell to migrate along a chemical gradient of a factor such as a cytokine that attracts the neural precursor cell. The chemical gradient may be created by secretion of one or more factors from a glioma cell. Thus, TMEM18 should direct migration along a gradient that is created by any cell that secretes a neural precursor cell chemoattractant factor.

Accordingly, in the present method, augmentation of TMEM18 levels in a neural precursor cell may enhance the migration of the neural precursor cell toward any cell that secretes a neural precursor cell chemoattractant factor. A “neural precursor cell chemoattractant factor” refers to any molecule, including a cytokine, that when placed in a concentration gradient causes migration of a neural precursor cell along the gradient toward the increasing concentration of the molecule.

Known factors that attract neural precursors include Stromal cell Derived Factor, Monocyte Chemoattractant Protein, Cytokine Stem Cell Factor, Epidermal Growth Factor, Vascular Endothelial Growth Factor, Stem Cell Factor or Fibroblast Growth Factor.

The cell that secretes the factor may produce the factor either naturally, as the result of carcinogenic or tumourogenic events within the cell or as a result of genetic modification, for example transformation or transfection of the cell with a nucleic acid molecule that directs expression of such a factor. As well, the cell that secretes the factor may produce the factor as the result of an immunogenic response to a tumour cell. Neural precursor cells have also been shown to migrate toward wound or injury sites within the brain; upon injection into brain tissue, neural precursor cells will tend to migrate to damaged areas and may then differentiate into specific neural cell types, such as neurons and glial cells that support neurons. Thus the cell that secretes the factor may also be a cell at a wound or injury site, including a site of neural tissue degeneration such as seen in neurodegenerative disorders, including neurodegenerative disorders including Parkinson's disease, damage from stroke, amyotrophic lateral sclerosis (ALS) and Huntington's disease.

In the present method, migration of a neural precursor cell is enhanced by augmented levels of TMEM18. The neural precursor cell may express endogenous TMEM18, meaning the cell has a native gene that encodes and expresses TMEM18. Thus, “augment”, “augmenting” or “augmentation” as used herein refers to increasing the levels of TMEM18 within the neural precursor cell above the levels normally seen in the cell in the absence of augmentation.

Augmenting the levels of TMEM18 includes modulating the expression levels of native endogenous TMEM18 by chemical or by genetic methods, including by exposure to a chemical or compound that increases expression of TMEM18 or by introducing a nucleic acid molecule or expression cassette encoding a regulatory factor that results in increased expression of native TMEM18 in the neural precursor cell.

Augmenting the levels of TMEM18 in a neural precursor cell also includes genetically modifying the neural precursor cell to include a nucleic acid molecule encoding TMEM18, including an expression cassette comprising the TMEM18 coding region. It will be understood that for TMEM18 to be expressed in the genetically modified neural precursor cell, the nucleic acid molecule will contain the coding region of TMEM18 operably linked to the necessary regulatory regions required to effect expression, including a suitable native or heterologous promoter region and optionally enhancer elements. As stated above, the neural precursor cells may already express TMEM18, and therefore the nucleic acid molecule encoding TMEM18 may be designed to express TMEM18 at levels above the natural levels of expression in the neural precursor cell.

Genetic modification can be achieved using molecular biology and cloning methods known in the art. For example, the precursor cell may be transformed or transfected with a vector designed to express TMEM18 from a native or heterologous promoter, and the promoter may be a constitutive, transient or inducible promoter, and may direct expression at basal or heightened levels of expression. Suitable vectors include bacterial plasmids or viral vectors including viral genomes. For example, a baculoviral, a retroviral, a lentiviral or an Adenoviral vector may be used.

The TMEM18 protein that is expressed using genetic modification techniques may be any TMEM18 protein that, when expressed in a neural precursor cells at augmented levels, that is to raise the overall level of TMEM18 in the neural precursor cell above levels seen with native expression of TMEM18 within that cell, enhances the ability of the neural precursor cell to migrate toward a cell that secretes a neural precursor chemoattractant factor.

Human TMEM18 protein (as described in NCBI accession no. NP_{—}690047.2) has the amino acid sequence:

[SEQ ID NO.: 1]

MPSAFSVSSFPVSIPAVLTQTDWTEPWLMGLATFHALCVLLTCLSSRSYR
LQIGHFLCLVILVYCAEYINEAAAMNWRLFSKYQYFDSRGMFISIVFSAP
LLVNAMIIVVMWVWKTLNVMTDLKNAQERRKEKKRRRKED

Mouse TMEM18 protein (as described in NCBI accession no. NP_{—}742046.1) has the amino acid sequence:

[SEQ ID NO.: 2]

METDWTEPWLLGLLAFHLLCLLLTCFSSQRYKLQIGHFLCLVVLVYSAEY
INEVAAVNWRLFSKYQYFDSRGMFISLVFSAPLLFNAMLIVIMWVRKTLT
VMTDLKTLQEERKERRRRRKEE

Rat TMEM18 protein (as described in NCBI accession no. NP_{—}001007749.1) has the amino acid sequence:

[SEQ ID NO.: 3]

MASPYSVRVFPVSIPAVIMETDWTEPWLLGLLAFHLLCLLLTCFSAQRYK
LQIGHFLCLVVLVYCAEYINEVAAMNWRLFAKYQYFDSRGMFISLVFSAP
LLFNAMVIVIMWVRKTLTVMSDLKNLQERRKERKRRRKEE

Dog TMEM18 protein (as described in NCBI accession no. XP_{—}848731.1) has the amino acid sequence:

[SEQ ID NO.: 4]

MCEWCCWQRPYAYLLSARDDFRCEGYGGPTKLRAPSLCITAPVEAACARV
ADPLSSSNGRLPARHRERSPRNFKLTGDVSVQRGLEHRKTGGKLPSQEAR
VPACLLLHHRGREGAADTLPQDHEWAVFCEWRLQERLARARARTTGASVT
VPIPAILGVHRPVTQTHRVSPSGLAVAQRPPRSQELCGLQHPARERSGGD
ALHERVVPRPGACCHLLPPGETATGPVHTGSPTPVRLGTRAGQESGPGPN
TRDTSHLRGAPFRNPTCRDAVPPPWGPLLRRVLPRLRLGPRPQTDWTEPW
LLGLAVFHVLCLLLTCLSSQRYKLQVGHFLCLVILVYCAEYINEIAAMNW
RLFSKYQYFDSRGMFISIVFSAPLLLNAMIIVILWVRKTLNVMTDLKTLQ
EKRRERKRKEE

Chicken TMEM18 protein (as described in NCBI accession no. NP_{—}001012716.1) has the amino acid sequence:

[SEQ ID NO.: 5]

MVSLAIWASSREASAVRMRSVAVAMEQPLHGPPGLSTILARTDWAEPWLL
GLAGFHVLCFLLTCFSFQHYRVQIGHFLCMVCLVYCAEYINELAAMNWRL
FSKYQYFDSRGMFISLVFSAPLLVNTIIIVVNWVYRTLNVMTELKTLQQR
IKAEKDKKK

A search of National Center of Biotechnology Information (NCBI) search program for homologous protein sequences (Homologene) also located homologous sequences from fruitfly (D. melanogaster) to rice (O. sativa).

Thus, “TMEM18 protein” as used herein refers to any TMEM18 protein and includes homologues of TMEM18, and any derivative, variant, or fragment thereof that is capable of enhancing migration of a neural precursor cell toward a glioma cell or a cell that secretes a neural precursor cell chemoattractant factor. A polynucleotide sequence or polypeptide sequence is a “homologue” of, or is “homologous” to, another sequence if the two sequences have substantial identity over a specified region and the functional activity of the sequences is conserved (as used herein, the term ‘homologous’ does not infer evolutionary relatedness). Two polynucleotide sequences or polypeptide sequences are considered to have substantial identity if, when optimally aligned (with gaps permitted), they share at least about 50% sequence identity, or if the sequences share defined functional motifs. In alternative embodiments, optimally aligned sequences may be considered to be substantially identical (i.e. to have substantial identity) if they share at least 60%, 70%, 75%, 80%, 85%, 90%, 95%, 96%, 97%, 98%, 99 identity over a specified region. An “unrelated” or “non-homologous” sequence shares less than 40% identity, though preferably less than about 25% identity, with a polypeptide or polynucleotide of the invention over a specified region of homology. The terms “identity” and “identical” refer to sequence similarity between two peptides or two polynucleotide molecules. Identity can be determined by comparing each position in the aligned sequences. A degree of identity between amino acid sequences is a function of the number of identical or matching amino acids at positions shared by the sequences, i.e. over a specified region. Optimal alignment of sequences for comparisons of identity may be conducted using a variety of algorithms, as are known in the art, including the ClustalW program, available at http://clustalw.genome.ad.ip, the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/).

A variant or derivative of TMEM18 refers to a fragment, either alone or contained in a fusion or chimeric protein, which retains the ability to enhance migration of a neural precursor cell toward a glioma cell or a cell that secretes a neural precursor cell chemoattractant factor, or a TMEM18 protein that has been mutated at one or more amino acids, including point, insertion or deletion mutation, but still retains the ability to direct migration of a neural precursor cell toward a glioma cell or a cell that secretes a neural precursor cell chemoattractant factor, as well as non-peptides and peptide mimetics which possess the ability to mimic the biological activity of TMEM18. A variant or derivative therefore includes deletions, including truncations and fragments; insertions and additions, including tagged polypeptides and fusion proteins; substitutions, for example conservative substitutions, site-directed mutants and allelic variants; and modifications, including peptoids having one or more non-amino acyl groups (q.v., sugar, lipid, etc.) covalently linked to the peptide and post-translational modifications. As used herein, the term “conserved amino acid substitutions” or “conservative substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing. Conservative changes can also include the substitution of a chemically derivatised moiety for a non-derivatised residue, for example, by reaction of a functional side group of an amino acid.

Variants and derivatives can be prepared, for example, by substituting, deleting or adding one or more amino acid residues in the amino acid sequence of a TMEM18 protein or fragment thereof, and screening for biological activity. Preferably, substitutions are made with conservative amino acid residues, i.e., residues having similar physical, biological or chemical properties. A skilled person will understand how to make such derivatives or variants, using standard molecular biology techniques and methods, described for example in Sambrook et al. ((2001) Molecular Cloning: a Laboratory Manual, 3^{rd} ed., Cold Spring Harbour Laboratory Press), and how to test such derivatives or variants for their ability to direct migration of a neural precursor cell toward a glioma cell or a cell that secretes a neural precursor cell chemoattractant factor including using techniques as described in the Examples set out herein.

In various embodiments, TMEM18 comprises an amino acid sequence of any one of [SEQ ID NO.: 1] to [SEQ ID NO. 5]. In particular, TMEM18 comprises the amino acid sequence of [SEQ ID NO.: 1].

In various embodiments, TMEM18 consists essentially of an amino acid sequence of any one of [SEQ ID NO.: 1] to [SEQ ID NO. 5]. In particular, TMEM18 consists essentially of the amino acid sequence of [SEQ ID NO.: 1]. As used herein, “consists essentially of” or “consisting essentially of” means that the sequence includes one or more amino acids at one or both ends of the described sequence, but that the additional amino acids do not materially affect the function of the TMEM18 protein to direct the migration of a neural precursor cell toward a glioma or toward a cell that secretes a neural precursor cell chemoattractant factor. For example, the TMEM18 protein consisting essentially of one of the above-mentioned sequences may have one, two, three, five or ten amino acids at one or both ends of the described sequence, provided that such a protein still possesses the ability to direct migration of a neural precursor cell toward a glioma cell or a cell that secretes a neural precursor cell chemoattractant factor.

In various embodiments, TMEM18 consists of an amino acid sequence of any one of [SEQ ID NO.: 1] to [SEQ ID NO. 5]. In particular, TMEM18 consists of the amino acid sequence of [SEQ ID NO.: 1].

Thus, by augmenting levels of TMEM18 in a neural precursor cell, including by genetically modifying the neural precursor cell by transfecting the cell with an expression vector that directs expression of TMEM18, the ability of the neural precursor cell to migrate along an increasing gradient of a chemoattractant factor is enhanced. “Enhancing” or “enhancement” of migration refers to increased rate of migration by a neural precursor cell as compared to the rate in the absence of augmented levels of TMEM18. Enhancing or enhancement of migration also refers to increasing the proportion of cells within a neural precursor cell population that will migrate as compared to the proportion of the population that migrates in the absence of augmented levels of TMEM18.

The neural precursor cell may be cultured in vitro in order to express TMEM 18 at augmented levels, under conditions and for a time suitable to increase the TMEM18 levels in the neural precursor cell. As will be appreciated, the neural precursor cells may be grown in vitro in an appropriate growth medium and at a temperature and for a time that generally maintains the neural precursor cell and which may allow for expansion of the neural precursor cell population, if desired. The growth conditions may be free from chemoattractant factors that attract neural precursor cells. Where an inducible promoter is used in an expression vector to effect the augmentation of TMEM18 levels, the growth conditions will include any conditions including any chemicals or compounds, temperature, light wavelength or level, necessary to induce expression of the TMEM18 gene.

The method may further include delivering the neural precursor cell with augmented TMEM18 levels to a cell population that includes a cell that secretes a neural precursor chemoattractant factor as described above. In one embodiment, the cell population to which the neural precursor cell is delivered includes a glioma cell. In another embodiment, the cell population to which the neural precursor cell is delivered includes a cell at a site of neural degeneration, such as a site of neural degeneration in a neurodegenerative disorder, including Parkinson's disease, damage from stroke, amyotrophic lateral sclerosis (ALS) and Huntington's disease.

The cell population to which the neural precursor cell is delivered may be an in vitro cell culture or may be an in vivo cell population within a subject.

Thus, when the neural precursor cell having augmented levels of TMEM18 is delivered to an in vivo cell population, the presently described method includes a method to treat a neural disorder, including a glioma or a neurodegenerative disorder.

The neural disorder may be any disorder, disease, wound, injury or damage to neural tissue that may be treated or repaired by administration of a neural precursor cell, and may include a glioma or a neurodegenerative disorder.

The glioma may be any glioma, including a glioma as described above. That is, the glioma may be a grade I-IV glioma, including a glioblastoma multiforme.

The neurodegenerative disorder may be any disorder characterized by deterioration of neural tissue, and includes Parkinson's disease, damage from stroke, amyotrophic lateral sclerosis (ALS) and Huntington's disease.

“Treating” a glioma or a neural degenerative disorder refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total). “Treating” can also mean prolonging survival of a subject beyond that expected in the absence of treatment. “Treating” can also mean inhibiting the progression of disease, slowing the progression of disease temporarily, although more preferably, it involves halting the progression of the disease permanently.

The subject may be any subject in need of treatment, including in need of treatment of a glioma or a neurodegenerative disorder. The subject may be any animal, including a mammal, particularly a human.

The neural precursor cell is administered to the subject by delivery to a site within the vicinity of the cell that secretes a neural precursor cell chemoattractant factor, including a glioma cell or a cell a site of neural tissue degeneration or within the vicinity of such a cell and within a distance to allow the neural precursor cells to travel to the site of the glioma cell or the cell a site of neural tissue degeneration. In vivo delivery may be accomplished using methods known in the art, including by surgical implantation or by injection.

An effective amount of neural precursor cells having augmented levels of TMEM18 are administered to the subject. The term “effective amount” as used herein means an amount effective, at dosages and for periods of time necessary to achieve the desired result, for example, to treat the glioma or neurodegenerative disorder.

The number of total neural precursor cells to be administered will vary, depending on several factors, including the severity and size of the glioma or the site of neural tissue degeneration, the extent to which the glioma has invaded surrounding healthy tissue, the type of cell that is administered, the mode of administration, and the age and health of the subject.

It will be appreciated that the neural precursor cells having augmented levels of TMEM18 may be administered to treat a glioma or a neurodegenerative disorder in combination with other treatments or therapies, including drug therapy, chemotherapy, radiation therapy and surgery.

When administered in vivo, the neural precursor cell may also be modified to include a marker molecule to allow for detection or visualization of the neural precursor cell once it has migrated toward a glioma cell or site of neural tissue degeneration within the brain of a subject. For example, the neural precursor cell may be modified to include a radioactive label or the neural precursor cell may be genetically modified to include one or more nucleic acid molecules encoding a marker protein or peptide, for example a nucleic acid molecule that encodes green fluorescent protein, yellow fluorescent protein, red fluorescent protein or luciferase, including as part of a fusion protein.

Alternatively, the neural precursor cell may be modified to include one or more additional therapeutic agents that are to be delivered to the site of a glioma cell or the site of neural tissue degeneration within a subject. For example, the neural precursor cell may be genetically modified to include a nucleic acid molecule encoding a therapeutic agent, such as a protein to be secreted at the site of a glioma cell or the site of neural tissue degeneration. It will be understood that the nucleic acid will comprise the coding region for a therapeutic protein or peptide as well as necessary regulatory regions required to effect expression of the therapeutic protein or peptide in the cell. Such regulatory regions include a suitable promoter region, such as a native or heterologous promoter, as well as, optionally, enhancer elements. For example, a vector, including a vector used to express TMEM18 in the neural precursor cell, may be designed to express a therapeutic protein or peptide.

A therapeutic protein or peptide is a protein or peptide, that when expressed in the precursor cell, has a therapeutic effect on the precursor cell or the cell that secretes the neural precursor cell chemoattractant factor, or which effects a desired result within the precursor cell or the cell that secretes the neural precursor cell chemoattractant factor. For example, the neural precursor cell may be modified to secrete a cytokine that directs immune cells to the site of a glioma, such as for example interleukin-23, or may be modified to express a suicide gene, for example the Herpes Simplex virus thymidine kinase gene.

Although the above-treatment method has been described in relation to treatment of glioma or neurodegenerative disorder, it will be appreciated that the method may be adapted to treat any disorder in which the migration of neural precursor cells along a chemoattractant factor gradient can be exploited to delivery neural precursor cells to a site within neural tissue where treatment is desired.

In addition to the above described methods, there is also contemplated a neural precursor cell having augmented levels of TMEM18, as described above. In certain embodiments, the neural precursor cell comprises an expression vector encoding TMEM18.

Thus, there is presently provided a neural precursor cell comprising a recombinant nucleic acid molecule encoding TMEM18, and which neural precursor cell is therefore genetically modified to express augmented levels of TMEM18. The neural precursor cell may further comprise a marker molecule. The neural precursor cell may further comprise a nucleic acid molecule, including an expression vector, encoding one or more therapeutic proteins or peptides.

To aid in administration of such a neural precursor cell to a subject, such as a subject in need of treatment of a glioma or a neurodegenerative disorder, a neural precursor cell having augmented levels of TMEM18 may be formulated as an ingredient in a pharmaceutical composition.

Therefore, there is provided a pharmaceutical composition comprising a neural precursor cell having augmented levels of TMEM18. The pharmaceutical composition may further include a pharmaceutically acceptable diluent or carrier. The invention in one aspect therefore also includes such pharmaceutical compositions for use in treating a glioma or a neurodegenerative disorder. The compositions may routinely contain pharmaceutically acceptable concentrations of salt, buffering agents, preservatives and various compatible carriers. For all forms of delivery, the neural precursor cell may be formulated in a physiological salt solution.

The pharmaceutical compositions may additionally contain other therapeutic agents useful for treating a glioma or a neurodegenerative disorder. In addition, the pharmaceutical composition may contain growth factors or cellular factors that facilitate cell survival and induce proliferation or differentiation of the neural precursor cell when delivered to the site of the glioma or neural tissue degeneration in a subject.

The proportion and identity of the pharmaceutically acceptable diluent or carrier is determined by the chosen route of administration, compatibility with live cells, and standard pharmaceutical practice. Generally, the pharmaceutical composition will be formulated with components that will not kill or significantly impair the biological properties of the live neural precursor cells.

The pharmaceutical composition can be prepared by known methods for the preparation of pharmaceutically acceptable compositions suitable for administration to subjects, such that an effective quantity of the neural precursor cells and any additional active substance or substances is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the neural precursor cells, in association with one or more pharmaceutically acceptable vehicles or diluents, and contained in buffer solutions with a suitable pH and iso-osmotic with physiological fluids.

Solutions of the neural precursor cells accordingly may be prepared in a physiologically and pharmacologically suitable buffer. Under ordinary conditions of storage and use, these preparations contain a preservative to prevent the growth of microorganisms, and that will maintain the live state of the cells. A person skilled in the art would know how to prepare suitable formulations. Conventional procedures and ingredients for the selection and preparation of suitable formulations are described, for example, in Remington's Pharmaceutical Sciences and in The United States Pharmacopeia The National Formulary (USP 24 NF19) published in 1999.

The pharmaceutical composition may be administered to a subject in a variety of forms depending on the selected route of administration, as will be understood by those skilled in the art. The composition of the invention may be administered surgically or by injection to the desired site.

In different embodiments, the composition is administered by injection (subcutaneously, intravenously, intramuscularly, etc.) directly at a desired site, for example in the vicinity of a glioma or neural tissue degeneration within the brain of a subject, including for example intercranial injection.

The dose of the pharmaceutical composition that is to be used depends on the particular glioma or neurodegenerative disorder being treated, the severity of the condition, individual patient parameters including age, physical condition, size and weight, the duration of the treatment, the nature of concurrent therapy (if any), the specific route of administration and other similar factors that are within the knowledge and expertise of the health practitioner. These factors are known to those of skill in the art and can be addressed with minimal routine experimentation.

The present methods and uses are further exemplified by way of the following non-limited examples.

### EXAMPLES

### Example 1

This study was designed to identify new proteins that could be used in bioengineering of neural precursor cells to achieve an improved ability to trace glioma cells or cells that secrete chemoattractant factors that attract neural precursor cells. This genetic modification could then facilitate the use of neural precursor cells as disease treatment vectors able to reach scattered disease sites, including scattered glioma cells.

A cDNA library screening method was used to discover new proteins that can, when overexpressed, specifically increase neuronal stem cell migration toward glioma cells in transwell migration assays. A previously uncharacterised protein Transmembrane protein 18 (TMEM18) emerged from the screen as a potential candidate to increase the glioma tropism of neural precursor cells. TMEM18 was then characterised to be an endogenous regulator of general cell motility and that it indeed can confer on neural precursor cells an improved preference for glioma cell-directed migration.

Materials and Methods

Cells and viruses: NT2, U87MG, H4, and NIH3T3 cell lines were purchased from ATCC (American Type Culture Collection, USA) and 293FT cells were purchased from Invitrogen. All the previous cell lines were maintained in DMEM supplemented with 10% fetal calf serum (Gibco), penicillin-streptomycin (Gibco), normoxin (Invivogen), and Non-essential amino acids (Gibco). C17.2 cells were maintained in DMEM supplemented with 10% fetal calf serum (Gibco), 5% horse serum (Gibco), penicillin-streptomycin (Gibco), normoxin (Invivogen), and Non-essential amino acids (Gibco).

Human Daudi cell cDNA library containing retrovirus supernatants were purchased from Stratagene and infection and maintenance was performed as recommended by the supplier. TMEM18 was cloned from Human Daudi cell cDNA library infected cells by PCR using following primers 5′ CACCATGCCGTCCGCCTTCTCTG [SEQ ID NO.: 6] and 5′ AAAGTCTTCTTTCCTTCTCCTTTTC [SEQ ID NO.: 7] and the PCR product was subsequently cloned into pLenti6/V5-TOPO vector (Invitrogen). Sequencing was used to ensure that the cloned sequence was correct (Research Biolabs). TMEM18A virus contained one amino acid mutation from alanine to threonine at position 103, which did not seem to have any effect in later experiments. Empty and TMEM18 lentiviruses were produced using VIRAPOWER™ Lentiviral Directional TOPO® Expression Kit and virus production, cell infection, selection, and cell maintenance was done as recommended by the manufacturer (Invitrogen).

Boyden chamber assay: In vitro migration of neural precursor cells toward glioma cells was examined using Boyden chamber assays. A migration kit from BD Falcon with 24-well cell culture plates was utilised. Each well of the plates was separated into two chambers by an insert membrane of 8-μm pores. One day before assays 50,000 glioma cells were seeded into each lower chamber. The next day cell culture medium in the lower chamber was removed and replaced with 500 μl of non-supplemented DMEM. Neural precursor cells (50,000 in 500 μl of non-supplemented DMEM) were then seeded into the upper chamber. After 12 or 24 hours of incubation at 37° C., migratory cells on the bottom of the insert membrane and non-migrating cells on the upper side of the membrane were dissociated by trypsination. These cells were subsequently lysed and stained using a CYQUANT™ cell proliferation assay kit (Molecular probes). Fluororescence was measured with a fluorescence plate reader (Tecan GENios pro). Results were expressed as ratios between the numbers of migratory to non-migrating cells in arbitrary units (AU). The average values and standard deviations of 6 to 12 wells are shown. Statistical analyses were done using Student's t-test.

cDNA library screen: One million of NT2 cells were infected with cDNA library retrovirus supernantants (described on section Cells and viruses) to yield 20% infection efficiency to ensure a proper presentation of all the cDNAs in the library. Cells were allowed to recover for 4 days after which they were passed through a transwell migration assay as described earlier. Migrating cells were collected and allowed to recover for 5 days after which they were passed through a second migration assay, collected and allowed to recover for 5 days. After a third migration assay both non-migrating and migrating cells were collected and left to recover.

Chromosomal DNA was collected from non-migrating and migrating cells using DNEASY™ kit (Qiagen) as recommended by the manufacture. Retrovirus imported sequences were recovered using virus PCR protocol and primers suggested in VIRAPORT™ manual (Stratagene). Same amount of chromosomal DNA was used in PCR for non-migrating and migrating cells. The success of the PCR was verified by running aliquot of the reactions on an agarose gel, before cloning the PCR products into pDrive using TA-cloning kit (Qiagen). DHα5 E. coli cells were transformed with the cloning products and plated. After overnight incubation, bacterial clones were picked and plasmid DNA was isolated, and then subsequently used in PCR using the same conditions as previously to isolate individual sequences, which were then sent to sequencing. Altogether 46 sequences from non-migrating cells and 70 sequences from migrating cells were sequenced (Research Biolabs).

RT-PCR: Cytoplasmic RNA was collected with RNEASY™ Kit (Qiagen) as recommended by the manufacturer. The concentration and the purity was verified before equal amounts of RNAs were used from all the samples for production of cDNA by reverse transcription using oligo-T-priming of Superscript III First-Strand Synthesis System™ (Invitrogen). Regular PCR for produced cDNAs was carried out using HotStart™ Taq system (Qiagen) as suggested by the HotStart™ Taq manual. Real-time PCR was done using Power Sybr Green PCR master mix and protocol (Applied Biosystems), primers for TMEM18 were 5′-ATGCCGTCCGCCTTCTCTG [SEQ ID NO.: 8] and 5′-GTCTTCTTTCCTTCTCCTTTTC [SEQ ID NO.: 9], and primers for beta-actin were 5′-TCATGTTTGAGACCTTCAA [SEQ ID NO.: 10] and 5′-GTCTTTGCGGATGTCCACG [SEQ ID NO.: 11]. Opticon 2 real-time PCR machine (Applied Biosystems) was used to run the PCR reactions, program for TMEM18 PCR was 10 minutes at 95° C., followed 45 cycles of 15 seconds at 95° C. followed by 1 minute at 68° C.; and program for beta-actin was 10 minutes at 95° C., followed by 40 cycles of 10 seconds at 95° C., 20 seconds at 55° C., and 20 seconds at 68° C. Real-time PCR results were displayed as a ratio of amount of TMEM18 mRNA to amount of beta-actin mRNA.

Scratch and cell proliferation assays: Cell were plated a day before the scratch assay to reach about 80% of confluency the day of assay. A layer of cells was removed using a pipet tip, fresh cell culture medium was changed, and light microscopy pictures of the cells were taken, and the exact location was marked on the cell culture plate. Cells were incubated for 24 hours before a second recording of the recovery of the scraped area.

For cell proliferation assay 5000 cells were plated on a 24-well plate, and cells from the well were collected by trypsination each day during a 4 day period. Collected cells were centrifuged and freezed at −20° C. The amount of cells in each sample was then counted using a CYQUANT™ cell proliferation assay kit (Molecular probes) as recommended by the supplier. The cells were counted using fluorescence plate reader (GENios pro, Tecan).

siRNA: Two sequences, 5′TCATCTTAGTCTACTGTGCTGAATA [SEQ ID NO.: 12] and 5′TGCTCACGCAGACGGACTGGACTGA [SEQ ID NO.: 13], were cloned into double promoter siRNA expression vector pFIV-H1/U6-PURO (System Biosciences) as recommended by the manufacturer's protocol. A siRNA sequence against luciferase provided in pFIV-vector cloning kit (System Biosciences) was prepared as a control. NT2 cells were plated to reach 90% confluence on the day of transfection of the siRNA expression plasmids, and plasmid DNA was transfected with Lipofectamine 2000 (Invitrogen) according to the manufacturer's protocol. Puromycin resistant cells were selected for 4 days after which cells were used for migration assay and for RT-PCR study.

Immunofluorescence: Antibody against TMEM18 was produced in rabbits against peptide 122-135: DLKNAQERRKEKKR [SEQ ID NO.: 14] (Biogenes GmbH) and used in 1:200 in immunofluorescence. Antibody against alpha-tubulin (ab7291) was purchased from Zymed and used in 1:100 in immunofluorescence. 4′,6-Diamidino-2-phenylindole (Molecular Probes), DAPI, was used in concentration of 2 nM. Secondary fluorescence antibodies were purchased from Jackson immunoresearch.

Cells were plated sparsely on a 24 well plate and the next day the cell culture medium was removed and cells were washed with PBS. Cells were then fixed in 4% paraformaldehyde for 10 minutes, washed with PBS, permeabilised in 0.2% triton-X-100 in PBS for 5 minutes, washed with PBS, and blocked in 1% BSA in PBS for 30 minutes. Antibodies were mixed with 1% BSA in PBS and incubated on cells for 30-60 minutes, after which the cells were washed three times with PBS and incubated with secondary antibodies in 1% BSA in PBS for 1 hour. Before studying the staining under fluorescence microscope cells were washed with PBS and stained briefly with DAPI.

In vivo cell migration assay: Rat C6 glioma cells (1 million cells in 5 μl) were injected into the right striatum of the rat brain (AP+1.0 mm, ML+2.5 mm, and DV −5.0 mm from bregma and dura) using a 10 μl Hamilton syringe connected with a 30-gauge needle at a speed of 0.5 μl/min. Three days later, 2.5 million cells (1.25 million green fluoresce dye-labeled TMEM18 over-expressing C17.2 cells and 1.25 million red fluoresce dye-labeled vector control C17.2 cells) were injected into the contralateral side of the rat brain. The brain samples were collected 2 week later for sectioning and examination. In the handling and care of animals, the Guidelines on the Care and Use of Animals for Scientific Purposes issued by National Advisory Committee for Laboratory Animal Research, Singapore was followed. The experimental protocols of the current study were approved by the Institutional Animal Care and Use Committee (IACUC), National University of Singapore and Biological Resource Center, the Agency for Science, Technology and Research (A* STAR), Singapore.

GFP fusion protein expression plasmids: GFP fusion protein expression plasmids were created as instructed in NT-GFP Fusion TOPO® Expression Kit manual (Invitrogen). TAT coding sequence was formed by annealing oligos 5′CAGCGCAAAAAACGCCGCCAGCGCCGCTAGA [SEQ ID NO.: 15] and 5′CTAGCGGCGCTGGCGGCGTTTTTTGCGCTGA [SEQ ID NO.: 16]. 15 amino acids from TMEM18 N-terminus was cloned by PCR using primers 5′TCAGTCTTCTTTCCTTCTCC [SEQ ID NO.: 17] and 5′AAGAATGCACAAGAGAGAAG [SEQ ID NO.: 18]. Before transfection into U87MG cell by Lipofectamine (Invitrogen) GFP fusion plasmids were sequenced.

Results

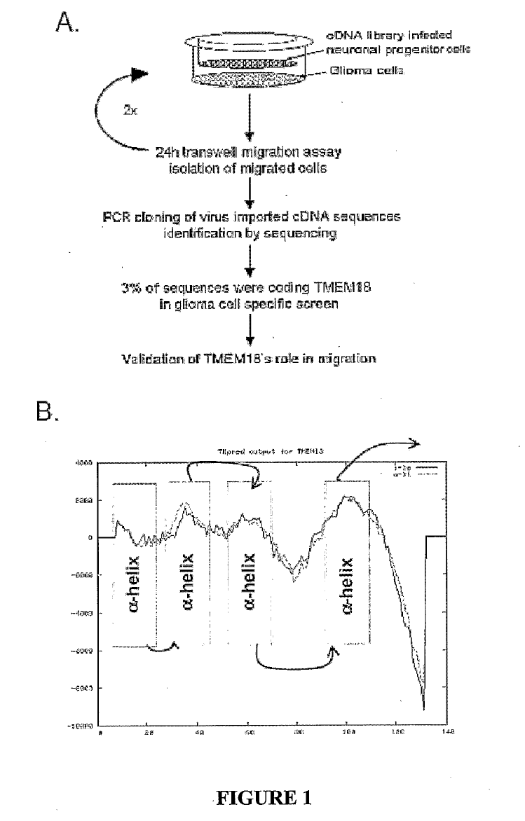

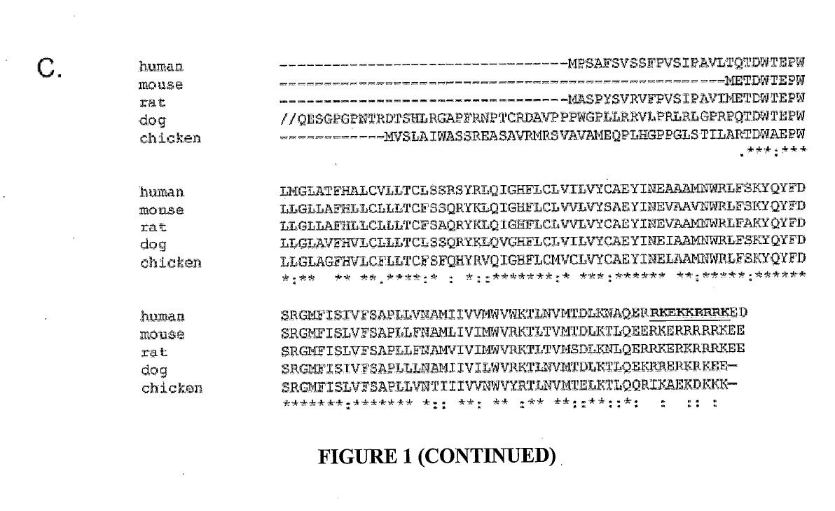

TMEM18 identified in a screen to discover genes to promote glioma-directed stem cell migration. Stem cells have been shown to be able to locate gliomas. A cDNA library screen was set up to identify new genes, which when overexpressed in stem cells, would further promote their ability to trace glioma cells. CDNA library was derived from lymphoma cell line, Daudi, and expressed from retrovirus vector. Human neuronal precursor cell line NT2 was infected with the cDNA library, and evaluated subsequently for their glioma-directed migration ability in a transwell cell migration assay. In transwell migration assay, cells, which are primed to migrate, squeeze through 8 micrometer pores into the opposite site of the transwell insert membrane, whereas non-migrating cells stay on the top of the membrane. Migrated cells were isolated and passed trough two more rounds of migration assay selection, after which the virus imported cDNAs were cloned by PCR and identified by sequencing, the protocol is summarised in FIG. 1. Non-migrating cells were used as controls for the analysis. Three percentages of the virus imported cDNA clones collected from the migrating cells were coding for TMEM18 gene, while no clone collected from the non-migrating cells had virus imported TMEM18 sequences. TMEM18 was thus a promising candidate gene for further analysis to validate its role in neural stem cell migration.

TMEM18 is a potential transmembrane protein with a C-terminal nuclear localization signal. TMEM18 is a previously uncharacterised protein with no published function attached to it so far. A search to find functional motifs from TMEM18 amino acid sequence using several web-based programs (ca.expacy.org/tools/) revealed no strong association, though weak potential sites for phosphorylation and N-myristoylation did appear. As the TMEM18 name indicates, the protein has four transmembrane spanning alpha-helixes as predicted by TMpred-program designed for identification of transmembrane proteins (Hofmann and Stoffel, 1993), although according to some other prediction programs the first membrane spanning part is less probable than the other three (FIG. 1B). Strongly hydrophilic C-terminus of the TMEM18 amino acid sequence had according to PredictNLS-program (Cokol et al, 2000) a probable nuclear localization signal (FIG. 1C, underlined sequence).

ClustalW-program (ch.EMBnet.org) was used to align TMEM18 protein sequences from human (NCBI accession no. NP_{—}690047.2), mouse (NP_{—}742046.1), rat (NP_{—}001007749.1), dog (XP_{—}848731.1), and chicken (XP_{—}419929.1) and revealed strong conservation across species (FIG. 1C), implying that TMEM18 possesses one or more key functions. Moreover, using National Center of Biotechnology Information (NCBI) search program for homologous protein sequences (Homologene), homologous sequences from fruitfly (D. melanogaster) to rice (O. sativa) were also found.

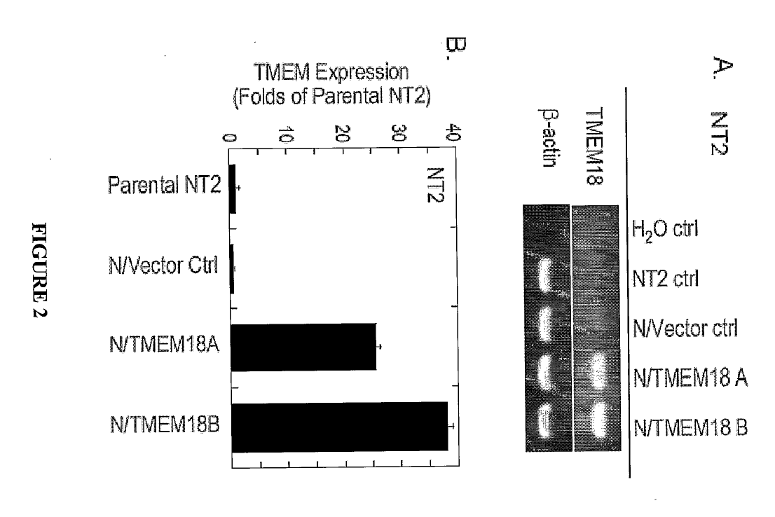

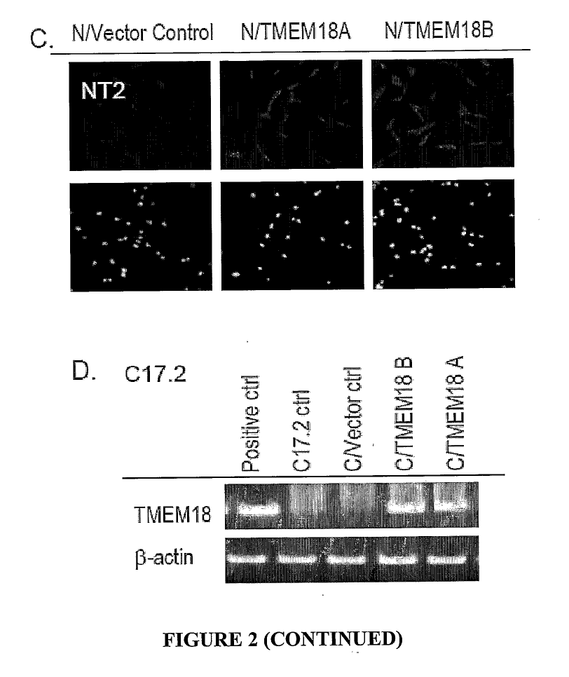

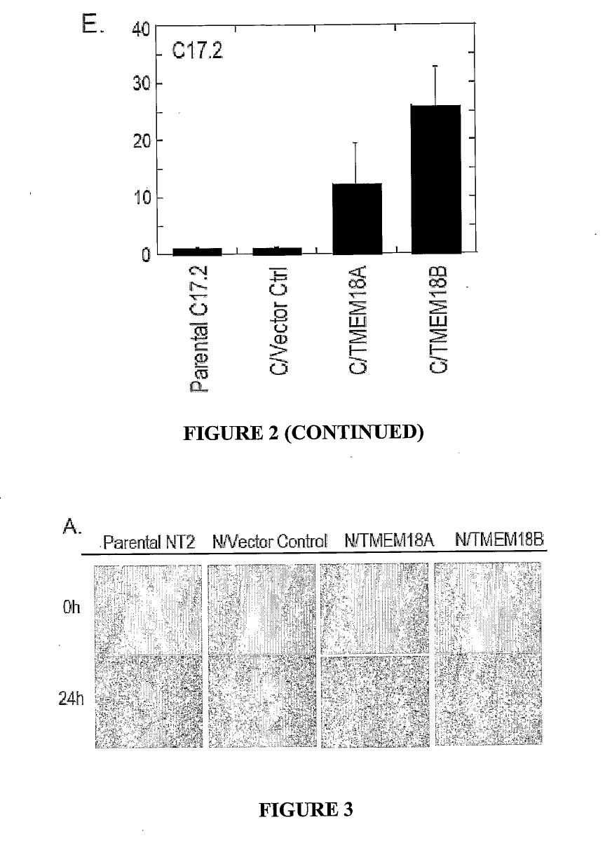

Overexpression of TMEM18 enhances the glioma-specific migration ability of neural precursor cells. TMEM18 overexpression was identified as a promoting factor for neural, stem cell migration toward glioma cells in our cDNA library screen. To verify the observation, the overexpression in huma neural precursor NT2 cells and a rat neural precursor C17.2 cells was investigated to determine if overexpression would dose-dependently affect the migration. Lentiviral vectors were used to create NT2 and C17.2 stable cells lines overexpressing TMEM18. The titer of lentivirus infection was controlled to have one virus per cells. Two populations of stable cells lines for each type of neural precursor cells that express different levels of TMEM18 were selected. TMEM18 overexpression was confirmed using RT-PCR (FIG. 2A for NT2 cells and FIG. 2D for C17.2 cells) and immunostaining (FIG. 2C). Quantified by real-time RT-PCR, NT2 TMEM18 cell lines A and B expressed TMEM18 mRNA about 25 and 38 times more than the controls, respectively (FIG. 2B) and the increases in C17.2 TMEM18 cell lines A and B were 10 and 25 fold, respectively (FIG. 2E).

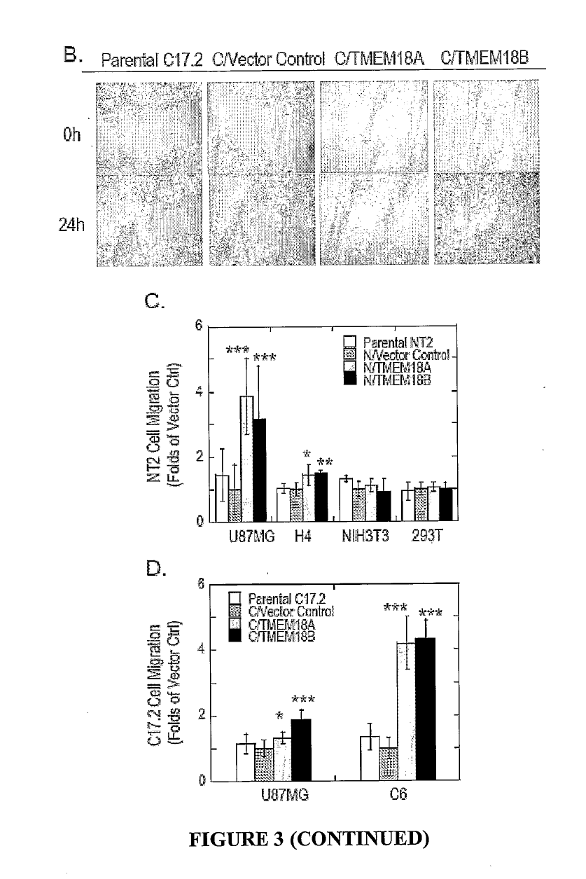

Scratch assays were first used to examine non-specific cell movement. Both NT2 and C17.2 populations that over-express TMEM18 showed overall increased migration capability, as demonstrated by moving into the scratched empty spaces much faster than parental cells or vector controls (FIGS. 3A and 3B). Cell proliferation assays confirmed that overexpression of TMEM18 did not affect the proliferation rates of different cells populations, thus excluding the possibility that the speed difference in covering the empty area in the scratch assay was due to increased propagation capacity of TMEM18 overexpressing cells.

Next, the movement of these cells lines towards U87 glioma cells (the tumour cell line used in the cDNA screen earlier) was examined, using transwell migration assays (FIGS. 3C and D). TMEM18 over-expressing NT2 and C17.2 cells displayed significantly higher migration capacities when compared with their parental cells and empty vector controls. These cells also responded to other glioma cell lines, H4 and C6, by displaying significant migration advantage. Interestingly, these TMEM18 over-expressing cells did not exhibit increased migration capacities when non-tumour cell lines, mouse fibroblast cell line NIH3T3 and human kidney cell line 293FT, were used in the transwell migration assays. Moreover, the amount of cells migrating to plain cell culture medium remained the same between TMEM18 overexpressing cells and the controls. Hence, the preference of TMEM18 overexpressing cells toward glioma cells implies a specialised role for TMEM19 in response to glioma secretary factors.

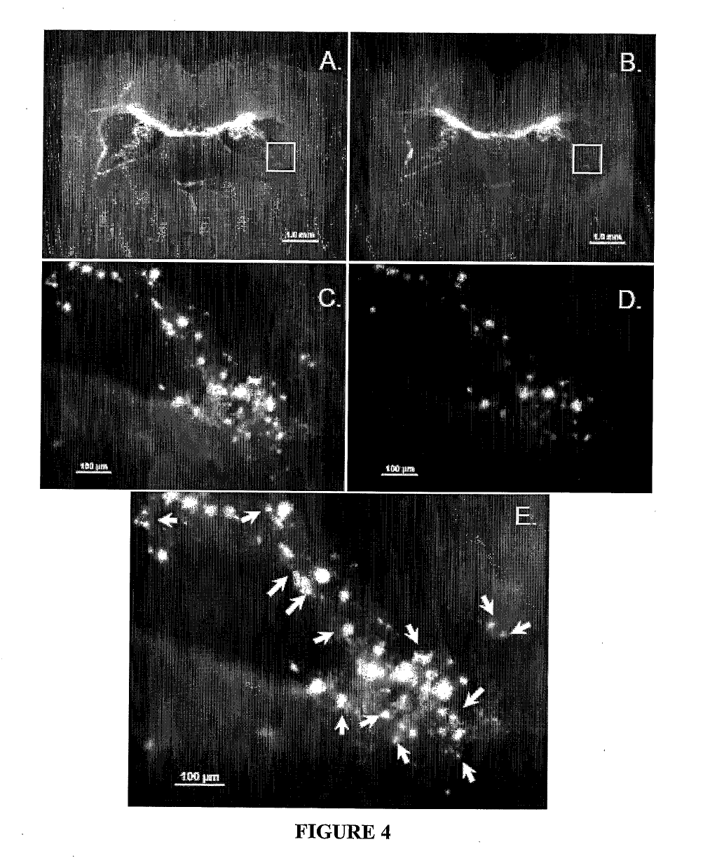

To supplement the above in vitro results, overexpression of TMEM18 was used to determine if the migration of C17.2 rat neural precursor cells towards gliomas in the brain could be improved. In a rat C6 glioma xenograft model, green fluorescence dye-labeled TMEM18 over-expressing C17.2 cells were injected together with red fluoresce dye-labeled vector control C17.2 cells on the side of the brain contralateral to the tumour inoculation site. Two weeks after the injection, the brain samples were collected for examination. As shown in FIG. 4, all cells migrated towards the tumour side and about half of them already crossed the middle line of the brain by week 2. At the front of the migrating cells, a significantly high number of green cells were observed, suggesting an improved migration of these TMEM18 over-expressing cells towards gliomas in the brain.

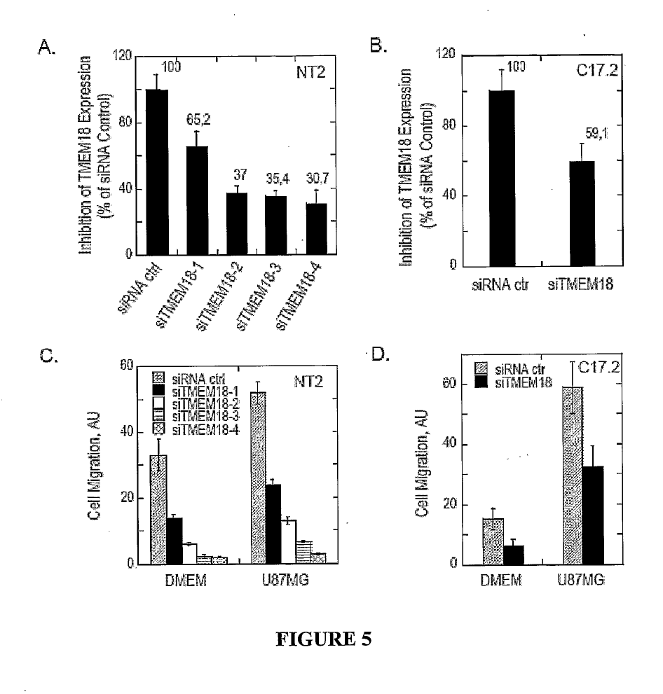

TMEM18 is critical for the migration of neural precursor cells. To verify whether endogenous levels of TMEM18 expression are involved in regulating neural stem cell migration, an RNA interference approach was used to shut down the expression of endogenous TMEM18 in NT2 cells. Puromycin resistant siRNA expression vector was constructed to express siRNA sequences against TMEM18 (two different sequences), or against luciferase. Two different transfections yielded four populations of NT2 cells with different levels of reduction of TMEM18 expression, ranging from 31, 35, 37 to 65% the original endogenous TMEM18 mRNA levels (FIG. 5A). A similar level of reduction was also observed in C17.2 cells (FIG. 5B). In a 24 hour transwell migration assay, siRNA against luciferase gene as a siRNA control did not seem to have major effects on the cell migration performance. On the other hand, siRNAs against TMEM18 did have a strong effect on the migration of NT2 and C17.2 cells. Reduction of the amount of TMEM18 mRNA into 60% of the normal levels lowered noticeable amount of migrating cells and diminishing the amount of TMEM18 expression to 31% of normal NT2 cell levels almost abolished cell migration ability completely (FIG. 5C). Interestingly, down-regulation TMEM18 reduced migration comparably both toward glioma cells and toward plain medium (FIGS. 5B and D), suggesting that TMEM18 is an important factor regulating general cell motility and is related to the overall migration capacity of neural stem cells.

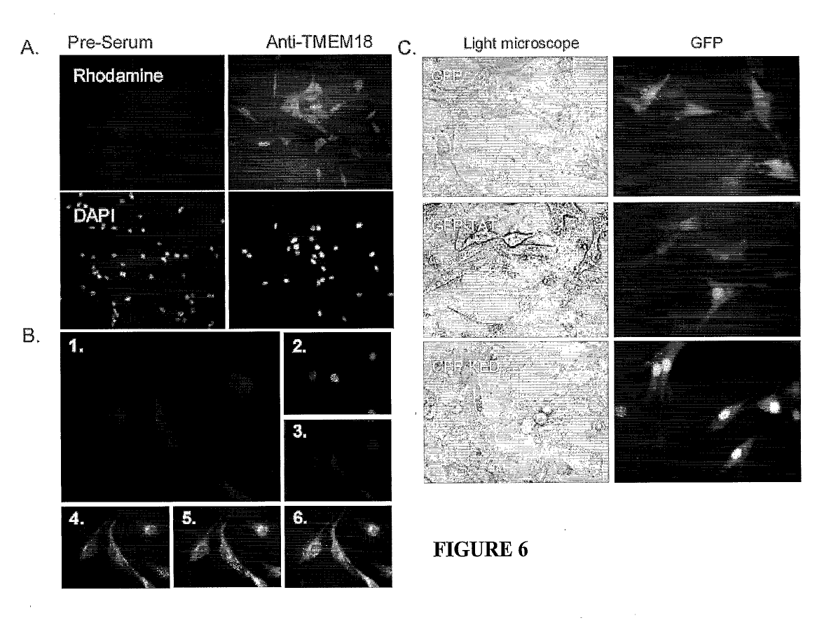

TMEM18 protein localises onto nuclear and cytoplasmic structures. To understand the mechanisms underlying the TMEM18 effects, the cellular localization of the TMEM18 protein was investigated. An antibody against TMEM18 C-terminal peptide was produced to uncover the cellular location of the TMEM18 protein. The specificity of the antibody was examined by immunofluorescence, in which cellular staining was compared between pre-immunization serum staining and TMEM18 antibody staining in controls and in TMEM18 over-expressing NT2 cell lines. Pre-immunization serum gave almost no signal, whereas TMEM18 antibody stained both control cells and in a slightly stronger manner the over-expressing samples (FIG. 6A). A closer look to the structures which the TMEM18 antibody (FIG. 6B1, in red) recognised in control NT2 cells revealed staining of nuclear membrane (a ring outlining the nucleus), insides of nucleus, and in restricted areas of cytoplasm (FIG. 6B). When the TMEM18 immunofluorescence staining was overlaid with nuclear staining (in blue) it confirmed that the ring structure was superimposable to nucleus (FIG. 6B3). In comparison with cytoskeletal structures stained with alpha-tubulin antibody (FIG. 6B4, in green) it was seen that TMEM18 localised only partly with the areas of tubulin network (FIGS. 6B5&6).

To test the function of the TMEM18 nuclear localization signal (NLS), GFP fusion protein approach was employed. Transfection of the control GFP plasmids in U87 cells lead to green fluoresce signals all over the cell (FIG. 6C). Noticeably, transfection with a plasmid vector encoding a hybrid protein composed of the last 15 N-terminal amino acids from TMEM18 (KED) and GFP resulted in a strong fluoresce signals in cell nucleus (FIG. 6C). The TMEM18 KED peptide appeared as effective as Tat peptide, a well-established nuclear localization signal peptide, in directing GFP into the nucleus.

Discussion

The cDNA library screen by itself could select cells having increased ability to migrate to glioma cells, improved unspecific cell movement, or enhanced proliferation rate. The last possibility was shown to be invalid, as TMEM18 overexpressing cells had similar cell cycle profile with the controls. When nonspecific cell movement was studied by a scratch assay it was seen that the TMEM18 cells were able to recover empty spaces faster than the control cells. Also, down-regulation of TMEM18 with siRNA reduced drastically cell migration ability in a transwell assay. Together these results show that TMEM18 affects cells' ability to move even without any spatial cues.

The transwell migration assays demonstrated specific migration increase of TMEM18 overexpressing cell lines toward glioma cells but not toward the plain cell culture medium, which was used as a control to detect the rate of unspecific migration. The TMEM18 over-expressing cells migrated in an identical manner with the control cell lines to kidney cell line, mouse fibroblast cell line, and to plain medium. No increased migration due to TMEM18 overexpression was coupled to spatial cues. I.e., even if an enhanced migration to target cells was obvious there was no change between TMEM18 cells and control cells. This demonstrates that TMEM18 does provide a glioma-derived cue sensing advantage for migration.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### REFERENCES

1. Tysnes, B. B., and Mahesparan, R. Biological mechanisms of glioma invasion and potential therapeutic targets. J Neurooncol, 53:129-47, 2001.
2. Mueller, M. M., Werbowetski, T., and Del Maestro, R. F. Soluble factors in glioma invasion. Acta Neurochir, 145: 999-1008, 2003.
3. Aboody, K. S., Brown, A., Rainov, N. G., Bower, K. A., Liu, S., Yang, W., Small, J. E., Herrlinger, U., Ourednik, V., Black, P. M., Breakefield, X. O., and Snyder, E. Y. Neural stem cells display extensive tropism for pathology in adult brain: evidence from intracranial gliomas. PNAS 97:12846-51, 2000
4. Benedetti, S., Pirola, B., Polio, B., Magrassi, L., Bruzzone, M. G., Rigamonti, D., Galli, R., Selleri, S., Di Meco, F., De Fraja, C., Vescovi, A., Cattaneo, E., and Finocchiaro, G. Gene therapy of experimental brain tumours using neural progenitor cells. Nat. Med., 6:447-50, 2000.
5. Flax, J. D., Aurora, S., Yang, C., Simonin, C., Wills, A. M., Billinghurst, L. L., Jendoubi, M., Sidman, R. L., Wolfe, J. H., Kim, S. U., and Snyder, E. Y. Engraftable huma neural stem cells respond to developmental cues, replace neurons, and express foreign genes. Nat. Biotechnol. 16:1033-9, 1998.
6. Arnhold, S., Hilgers, M., Lenartz, D., Semkova, I., Kochanek, S., Voges, J., Andressen, C., and Addicks, K. Neural precursor cells as carriers for a gene therapeutical approach in tumour therapy. Cell Transplant. 12:827-37, 2003.
7. Brown, A. B., Yang, W., Schmidt, N. O., Carroll, R., Leishear, K. K., Rainov, N. G., Black, P. M., Breakefield, X. O., and Aboody, K. S. Hum Gene Ther. 14:1777-85, 2003.
8. Allport, J. R., Shinde Patil, V. R., and Weissleder, R. Murine neuronal progenitor cells are preferentially recruited to tumour vasculature via alpha4-integrin and SDF-1alpha-dependent mechanisms. Cancer Biol Ther. 3:838-44, 2004.
9. Aarum, J., Sandberg, K., Haeberlein, S. L., and Persson, M. A. Migration and differentiation of neural precursor cells can be directed by microglia. PNAS. 100:15983-8. 2003
10. Tran, P. B., and Miller, R. J. Chemokine receptors: signposts to brain development and disease. Nat Rev Neurosci. 4:444-55, 2003.
11. Ehtesham, M., Yuan, X., Kabos, P., Chung, N. H., Liu, G., Akasaki, Y., Black, K. L., and Yu, J. S. Glioma tropic neural stem cells consist of astrocytic precursors and their migratory capacity is mediated by CXCR4. Neoplasia. 6:287-93, 2004.
12. Imitola, J., Raddassi, K., Park, K. I., Mueller, F. J., Nieto, M., Teng, Y. D., Frenkel, D., Li, J., Sidman, R. L., Walsh, C. A., Snyder, E. Y., and Khoury, S. J. Directed migration of neural stem cells to sites of CNS injury by the stromal cell-derived factor 1 alpha/CXC chemokine receptor 4 pathway. PNAS, 101:18117-22, 2004.
13. Widera, D., Holtkamp, W., Entschladen, F., Niggemann, B., Zanker, K., Kaltschmidt, B., and Kaltschmidt, C. MCP-1 induces migration of adult neural stem cells. Eur J. Cell Biol. 83:381-7, 2004
14. Erlandsson, A., Larsson, J., and Forsberg-Nilsson, K. Stem cell factor is a chemoattractant and a survival factor for CNS stem cells. Exp Cell Res. 301:201-10, 2004.
15. Sun, L., Lee, J., and Fine, H. A. Neuronally expressed stem cell factor induces neural stem cell migration to areas of brain injury. J Clin Invest. 113:1364-74. 2004.
16. Serfozo, P., Schlarman, M. S., Pierret, C., Maria, B. L., and Kirk, M. D. Selective migration of neuralized embryonic stem cells to stem cell factor and media conditioned by glioma cell lines. Cancer Cell Int. 6:1, 2006.
17. Boockvar, J. A., Kapitonov, D., Kapoor, G., Schouten, J., Counelis, G. J., Bogler, O., Snyder, E. Y., McIntosh, T. K., and O'Rourke, D. M. Constitutive EGFR signaling confers a motile phenotype to neural stem cells. Mol Cell Neurosci. 24:1116-30, 2003.
18. Schmidt, N. O., Przylecki, W., Yang, W., Ziu, M., Teng, Y., Kim, S. U., Black, P. M., Aboody, K. S., and Carroll, R. S. Brain tumour tropism of transplanted huma neural stem cells is induced by vascular endothelial growth factor. Neoplasia. 7:623-9, 2005.
19. Ziu, M., Schmidt, N. O., Cargioli, T. G., Aboody, K. S., Black, P. M., and Carroll R. S. Glioma-produced extracellular matrix influences brain tumour tropism of huma neural stem cells. J. Neurooncol. 79(2):125-33, 2006.
20. Larsen, M., Tremblay, M. L., and Yamada, K. M. Phosphatases in cell-matrix adhesion and migration. Nat Rev Mol Cell Biol. 4:700-11, 2003.
21. Cokol, M., Nair, R., and Rost, B. Finding Nuclear localization signals. EMBO Rep 1: 411-415, 2000.
22. Hofmann, K., and Stoffel, W. TMbase—A database of membrane spanning proteins segments Biol Chem Hoppe-Seyler 374:166, 1993.
23. Strausberg, R. L., Feingold, E. A., Grouse, L. H., Derge, J. G., Klausner, R. D., Collins, F. S., Wagner, L., Shenmen, C. M., Schuler, G. D., Altschul, S. F., Zeeberg, B., Buetow, K. H., Schaefer, C. F., Bhat, N. K., Hopkins, R. F., Jordan, H., Moore, T., Max, S. I., Wang, J., Hsieh, F., Diatchenko, L., Marusina, K., Farmer, A. A., Rubin, G. M., Hong, L., Stapleton, M., Soares, M. B., Bonaldo, M. F., Casavant, T. L., Scheetz, T. E., Brownstein, M. J., Usdin, T. B., Toshiyuki, S., Carninci, P., Prange, C., Raha, S. S., Loquellano, N. A., Peters, G. J., Abramson, R. D., Mullahy, S. J., Bosak, S. A., McEwan, P. J., McKernan, K. J., Malek, J. A., Gunaratne, P. H., Richards, S., Worley, K. C., Hale, S., Garcia, A. M., Gay, L. J., Hulyk, S. W., Villalon, D. K., Muzny, D. M., Sodergren, E. J., Lu, X., Gibbs, R. A., Fahey, J., Helton, E., Ketteman, M., Madan, A., Rodrigues, S., Sanchez, A., Whiting, M., Madan, A., Young, A. C., Shevchenko, Y., Bouffard, G. G., Blakesley, R. W., Touchman, J. W., Green, E. D., Dickson, M. C., Rodriguez, A. C., Grimwood, J., Schmutz, J., Myers, R. M., Butterfield, Y. S., Krzywinski, M. I., Skalska, U., Smailus, D. E., Schnerch, A., Schein, J. E., Jones, S. J., and Marra, M. A.; Mammalian Gene Collection Program Team. Generation and initial analysis of more than 15,000 full-length human and mouse cDNA sequences. PNAS, 99:16899-903, 2002.

1. A method of enhancing migration of a neural precursor cell toward a cell that secretes a neural precursor cell chemoattractant factor, the method comprising augmenting levels of TMEM18 in the neural precursor cell.

2. The method according to claim 1 wherein the cell that secretes a neural precursor chemoattractant factor is a glioma cell.

3. The method according to claim 1 wherein the cell that secretes a neural precursor chemoattractant is a cell at a site of neural tissue degeneration.

4. The method according to claim 1 wherein the TMEM18 comprises an amino acid sequence as set forth in any one of SEQ ID NO.: 1 to SEQ ID NO.: 5.

5. The method according to claim 1 wherein augmenting comprises increasing expression levels of native TMEM18 in the neural precursor cell.

6. The method according to claim 1 wherein augmenting comprises expressing recombinant TMEM18 from an expression vector in the neural precursor cell.

7. The method according to claim 1 wherein the neural precursor chemoattractant factor comprises Stromal cell Derived Factor, Monocyte Chemoattractant Protein, Cytokine Stem Cell Factor, Epidermal Growth Factor, Vascular Endothelial Growth Factor, Stem Cell Factor or Fibroblast Growth Factor.

8. The method according to claim 1 wherein the neural precursor cell is derived from an embryonic stem cell.

9. The method according to claim 1 wherein the neural precursor cell is derived from neural tissue.

10. The method according to claim 1 further comprising delivering the neural precursor cell to a cell population containing the cell that secretes the neural precursor chemoattractant factor.

11. The method according to claim 10 wherein the cell population is in vitro.

12. The method according to claim 10 wherein the cell population is in vivo.

13. The method according to claim 12 comprising delivering the neural precursor cell by surgical implantation or injection.

14. The method according to claim 10 wherein the neural precursor cell has been modified to include a marker molecule.

15. The method according to claim 10 wherein the neural precursor cell has been modified to include a therapeutic agent.

16. A neural precursor cell having augmented levels of TMEM18.

17. The neural precursor cell according to claim 16 wherein the neural precursor cell comprises an expression vector encoding TMEM18.

18. The neural precursor cell according to claim 17 wherein the TMEM18 comprises an amino acid sequence as set forth in any one of SEQ ID NO.: 1 to SEQ ID NO.: 5.

19. The neural precursor cell according to claim 16 further comprising an expression vector encoding a therapeutic protein or peptide.

20. The neural precursor cell according to claim 16 for treating a neural disorder.

21. A pharmaceutical composition comprising a neural precursor cell of claim 16.

22. (canceled)

23. (canceled)
